(19) United States
(12) Patent Application Publication
(10) Pub. No.: US 2009/0306567 A1
(43) Pub. Date: Dec. 10, 2009

(54) CERVICAL SPINE TRACTION APPARATUS AND METHOD

(76) Inventor: Donald W. Meyer, Fountain Valley, CA (US)

Correspondence Address: STETINA BRUNDA GARRED & BRUCKER, 75 ENTERPRISE, SUITE 250, ALISO VIEJO, CA, 92656, US

(21) Appl. No.: 12/135,753

(22) Filed: Jun. 9, 2008

(51) Int. Cl. A61F 5/00

(52) U.S. Cl. 602/33; 602/32; 602/36; 602/4

(57) ABSTRACT

A cervical traction method and apparatus for treating irregularities in the lateral curvature of the cervical region of the spine involves the application of traction load to induce an axially elongated or extension posture in the cervical region of the patient's spine. The patient is placed in a supine or seated position, and a traction halter is fitted about the head of the patient, with a traction sling also being optionally positioned about the cervical region of the patient. A traction load, which may have an intermittently varying magnitude, is exerted on one or more of the sling or halter to induce an axially elongated and/or extension posture in the cervical region. The axially elongated posture at least partially diminishes the lateral curvature of the cervical region of the spine in the patient, and the extension posture at least partially restores the lateral curvature to the cervical region of the spine in the patient, thereby reducing symptoms such as neck pain, upper back pain, headaches, and other symptoms of mechanical origin that result from irregular lateral curvature of the spine.

### CROSS-REFERENCE TO RELATED APPLICATIONS

Not Applicable.

### STATEMENT RE: FEDERALLY SPONSORED RESEARCH/DEVELOPMENT

Not Applicable

### BACKGROUND OF THE INVENTION

1. Technical Field

The present invention relates generally to an apparatus and method for achieving traction of the cervical region of the spine to treat conditions related to irregular lateral curvature of the cervical region, including cervicogenic pain symptoms of mechanical origin related to reduced or excessive cervical lordosis/extension and altered posture, such as abnormal forward or backward head translation.

2. Related Art

Proper anterior to posterior alignment and lateral curvature of the spine are known to be important to maintain good posture and function of the spine. FIGS. 1A-1C illustrate peer reviewed and published ideal lateral curvature of a spinal column 8, with FIGS. 1A and 1C showing the semi-circular curvature through the cervical region (C1-C7) 10, the elliptical curvature of the thoracic region (T1-T12) 13, and the lumbar region (L1-L5) 15. FIG. 1B illustrates the same spinal column 8 as viewed from the back of the individual, and showing the different regions of the spine 8. As show in FIGS. 1A and 1C, the thoracic region 13 of the spinal column 8 has an elliptical concave curvature as viewed from the front of the individual. In contrast the cervical region 10 (located in the region of the neck) has a semi-circular convex curvature, which convexity is echoed in the elliptical curvature of the lumbar region 15 (located in the region of the lower back). Improper anterior to posterior alignment and/or lateral curvature of regions of the spine can develop through accident or trauma or as a result of illness such as tumor or osteoporosis (bone density loss), from idiopathic causes, and/or may also occur in the normal process of aging, as in spondylosis (also known as degenerative joint disease). Such irregular curvature, alignment and posture of the spine 8 is often accompanied by reduced motion, pain and discomfort, which may severely reduce and impair the quality of life, and can even be disabling for such individuals. For example, improper lateral curvature of the cervical region 10 of the spine 8 may take the form of hypolordosis or kyphosis, which is a partial or complete loss or reversal of the natural curvature of the cervical region 10 that can cause pain and discomfort, such as headaches and neck/upper back pain. Another example is Forward Head Posture (FHP), which is a condition when the head is held at an abnormal posture of more than 15 millimeters forward of the shoulders. This occurs in approximately 66% of the population and is associated with a barrage of symptoms from headaches to Temporomandibular Joint (TMJ) disorders to increased nerve tension-related shoulder/arm/hand pain.

Various methods of treating pain in the cervical region resulting from structural irregularities of the spine are known in the art. For example, one method used for the treatment of pain in the neck region involves cervical traction, in which at least a portion of the cervical region of the spinal column is stretched to achieve axial distraction of the spine and reduce axial stresses on the discs and facet joints. Such cervical traction can be achieved through the use of conventional over-the-door static traction, where a person sits and wears a head halter that is attached to a water weight bag via a rope and two pulleys, the pulleys being mounted to a bracket attached to a top edge of a closed door. In a more recent method, traction of the cervical region of the spine is achieved by strapping a patient in a chair or on a semi-supine treatment/examining table, fitting a portion of the cervical spine with a traction strap, and exerting a static traction load to pull the strap and thereby transversely stretch the spine. In these methods, the static traction force applied to the body induces an extension posture in the cervical region of the spine that is maintained at an intensity and for a duration sufficient to induce musculoligamentous changes, thereby “remodeling” the spine into a more proper lateral curvature. This method is thus effective to increase and shape the curvature of the spine to combat conditions such as hypolordosis or kyphosis. Further description of this and other cervical traction methods are described in more detail in Harrison CBP Seminars, Inc. Publication, Chapter 6, the “History of Cervical Traction,” having a copyright date of 2004, which is herein incorporated by reference in its entirety.

A problem with conventional static, extension traction methods is that they often do not achieve proper remodeling of the spine within an acceptable time frame. For example, because the traction force applied during treatment is limited by the patient's tolerance level, proper treatment may require 30-60 repeated traction sessions in order to achieve the desired treatment outcome. This is a problem because the scheduling and coordinating of multiple sessions can be inconvenient for many patients, and also patient compliance with the treatment program may decrease when numerous sessions are involved. Also, the patients may continue to suffer from unacceptable levels of pain until the multiple required traction sessions have been completed. Yet another problem with conventional static extension traction methods is that most patients experience discomfort during prolonged static traction sessions due to the constant and unchanging tension exerted in the spine. The effectiveness of the treatment may also decrease over time as a result of the contracture or activation of muscles in response to the static traction force (i.e., “muscle-guarding”). Accordingly, there remains a need for a safe and efficient means of treating patients having an abnormal or irregular lateral curvature of the spine in the cervical region of the spine, without excessive pain or discomfort. A device that performs traditional cervical axial traction as well as a multiple number of the various one and two-directional methods of cervical extension traction would also be cost and space effective and would therefore be useful for the practitioner or therapist.

### BRIEF SUMMARY OF THE INVENTION

The present invention specifically addresses and alleviates the above-identified deficiencies in the art. In this regard, the present invention is directed to a cervical traction method for treating irregularities in a lateral curvature of a cervical region of a spine in a patient in need thereof, such as a method for treating irregularities in a lateral curvature of the cervical region. The method involves placing the patient in a supine or seated position and positioning a traction halter about the head of the patient, with a traction sling also being optionally positioned about the cervical region of the patient. A traction load is exerted on one or more of the sling or halter to induce at least one of an elongation and an extension posture in at least a portion of the cervical region of the spine. The elongation and/or extension posture at least partially restores proper curvature of the cervical region of the spine in the patient, thereby increasing range of motion of the cervical region and reducing symptoms such as neck pain, headaches, and other symptoms of mechanical origin that accompany abnormal or irregular spinal curvature.

In one version, the method of cervical traction is enhanced by exerting a traction load having intermittently varying magnitude, such as a non-zero magnitude that varies sinusoidally For example, the intermittent traction load can vary sinusoidally from a maximum to a minimum magnitude having a difference of about 1 lb to about 20 lbs. A frequency of variation of the intermittent traction load may be from about 5 cycles/min to about 20 cycles/min. The traction halter and optional traction sling can be positioned and pulled from angles that are selected to provide the desired axial and/or extension posture in the cervical region of the spine, such as an axial extension posture or a compression extension posture.

A traction apparatus capable of providing the cervical traction can include a traction halter that is sized and configured to fit about a head of a patient, a traction motor capable of exerting a traction load, an optional traction sling that is sized and configured to fit about the cervical region of the spine of the patient, and a load transfer line having a first end capable of being placed in mechanical communication with the traction motor and a second end capable of being placed in mechanical communication with either the traction halter or the traction sling. The load transfer line is capable of transferring the traction load from the traction motor to the traction halter or traction sling to induce at least one of an axial elongation and/or extension posture in the cervical region of the spine, thereby effecting traction of the cervical region of the spine. In one version, the traction apparatus includes a traction support having an L-frame with a laterally extending support arm that is configured to be capable of anchoring the load transfer line at a position above the patient. The traction support allows the traction halter and/or traction sling to be pulled in an upward direction upon exertion of the traction load.

The present invention is best understood by reference to the following detailed description when read in conjunction with the accompanying drawings.

### BRIEF DESCRIPTION OF THE DRAWINGS

These as well as other features of the present invention will become more apparent upon reference to the drawings wherein:

FIG. 1A is a schematic side view of a spinal column having normal curvature and showing the cervical, thoracic and lumbar regions of the spine;

FIG. 1B is a schematic back view of the spinal column of FIG. 1A;

FIG. 1C is a schematic side view showing the curvature of the spinal column of FIG. 1A;

FIGS. 2A and 2B are schematic front views of embodiments of a cervical traction apparatus comprising a traction motor capable of outputting an intermittent traction load according to the instant invention;

FIG. 2C is a schematic front view of an embodiment of a strap ratchet winch mechanism suitable for use with the cervical traction apparatus of FIGS. 2A and 2B.

FIGS. 3A-3H are schematic side views of embodiments of cervical traction apparatus having configurations for effecting cervical traction in the spine of a patient;

FIG. 4A is a schematic side view of a version of a traction motor capable of outputting a traction load having an intermittently varying magnitude;

FIG. 4B is schematic sectional front view of the traction motor of FIG. 4A; and

FIG. 5 is a schematic side view of a traction support arm having pulleys mounted thereon.

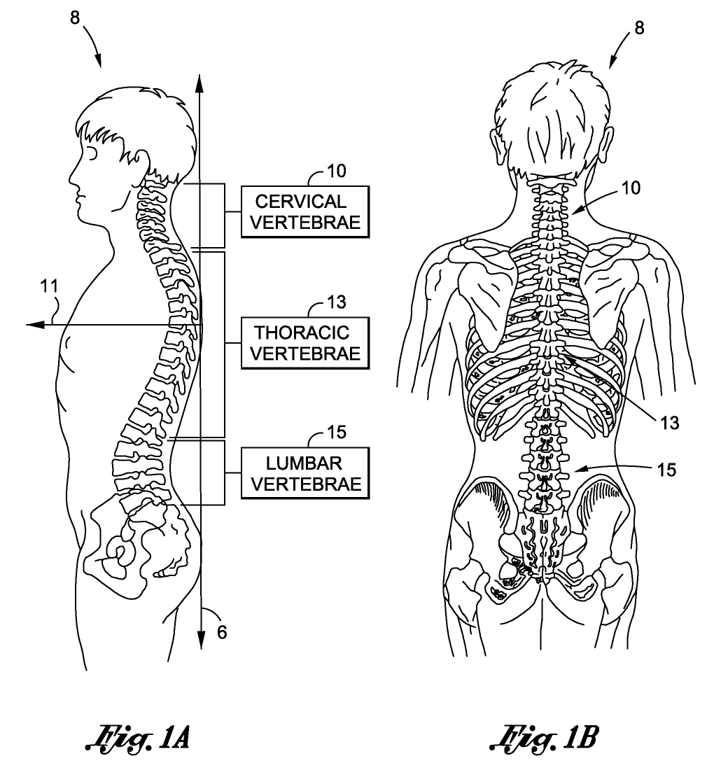

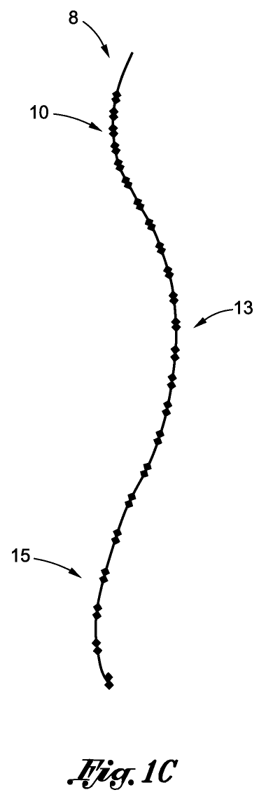

Common reference numerals are used throughout the drawings and detailed description to indicate like elements.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

A cervical spine traction method and apparatus has been discovered that is capable of treating irregularities in the curvature of the cervical region 10 of the spine 8 of the patient 25. The method involves exerting a one or two-directional traction load on at least a portion of the cervical region 10 of the spine 8 to induce a traction posture corresponding to at least one of an extension posture and an axially elongated posture in the cervical region 10. The extension and/or axially elongated posture is maintained at an intensity and for a duration sufficient to induce musculoligamentous changes in the cervical region of the spine, thereby “remodeling” the spine into a more proper alignment and at least partially restoring the normal lateral cervical curvature of the spine. The extension posture and/or axially elongated posture can include extension and/or elongation of one or more of an upper, lower or mid portion of the cervical region 10, according to the desired treatment. By “extension posture,” it is meant that the cervical spinal region is stretched to achieve axial and posterior distraction of the vertebrae of the spine 8, and/or that the spinal region is bent backward to extend at least some of the vertebrae of the spine into the shape of an anterior curve. For example, in axial extension of the cervical region 10 of the spine 8, the vertebrae of the spine are stretched apart from one another along a posterior directed, longitudinal axis 6 of the spine 8. In compression extension of the cervical region 10 of the spine 8, the cervical region of the spine (i.e., the neck) is bent backwards to curve the vertebrae anterior, with anterior stretching of the vertebrae and soft tissues on the front of the neck 64, and posterior compression of the vertebrae and soft tissues at the back of the neck 64. An “axially elongated posture” can also be one that elongates the cervical region 10 by stretching apart the vertebrae of the cervical region 10, such as by stretching axially along the longitudinal axis 6 of the spine. The traction method can involve inducing either an extension posture or an axially elongated posture, or may induce a traction posture that combines elements of extension with axial elongation of the cervical region 10 of the spine 8.

It has been further discovered that traction of the cervical region 10 of the spine 8 can be enhanced by the application of an intermittent traction load. The application of the intermittent traction load can be used to enhance intervertebral separation of the cervical region 10 of the spine 8 by reducing the “muscle-guarding” response and thereby increasing the patient comfort level and therefore the effectiveness of the traction, as well as possibly reducing the duration and frequency of cervical traction sessions necessary to restore a more normal cervical spinal structure/posture. By “intermittent traction load” it is meant that the traction load (i.e. traction force) that is applied to achieve cervical traction has a magnitude that varies over a selected duration, such as from a minimum traction load to a maximum traction load, as opposed to a “static” traction load that is applied at a continuous and unchanging magnitude. Without being bound by any theory, it is believed that the application of the intermittent traction load decreases the “muscle-guarding” response that is otherwise elicited in static traction methods, which improves intervertebral separation and patient comfort during the traction process and also increases the effectiveness of the treatment.

The intermittent traction load can be applied at magnitudes and frequencies that are selected according to factors such as the type and severity of the spinal condition being treated, as well as the age, body size and tolerance level of the patient, and the number and duration of traction sessions to be administered. The intermittent traction load is a non-zero load applied over a pre-selected period of time, and having a varying, non-zero magnitude. In one version, the intermittent traction load has a continuously varying magnitude that cycles from a minimum traction load level to a maximum traction load level at a pre-selected frequency. Such a continuously varying traction load could also be characterized as a having sinusoidally varying magnitude, and may be desirable to reduce “jarring” of the patient that might other wise occur with sudden or abrupt changes in traction load. As an example, the intermittent traction load may substantially continuously vary in magnitude from a minimum traction load to a maximum traction load having a difference in load poundage of from about 1 to about 20 lbs, such as from about 1 to about 7 pounds. The frequency of the traction load variation cycle may be from about 5 cycles/min to about 20 cycles/min, such as about 12 cycles/min. The rate of resting human respiration, approximately 12-13 cycles/min., can also be utilized and is recommended for improved patient relaxation. Alternatively, the frequency of the variation in the traction load and/or the minimum and maximum traction loads may be increased or decreased throughout the duration of the cervical traction session, or increased or decreased between sessions. For example, a maximum traction load applied in an initial traction session may be from about 7 to about 18 lbs, such as from about 10 to about 15 lbs, which maximum traction load can be increased with subsequent sessions but typically should not be allowed to exceed 40 lbs.

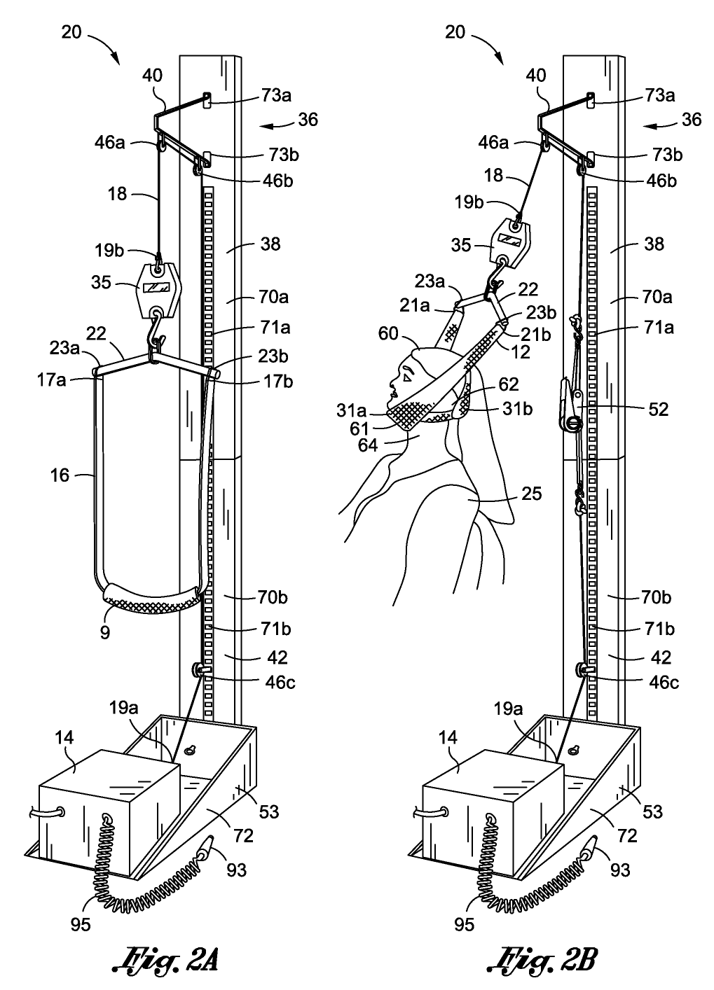

FIGS. 2A and 2B show embodiments of a cervical traction apparatus 20 capable of providing traction of the cervical region 10 of the spine 8 of a patient 25. The traction apparatus 20 comprises a traction halter 12 that is sized and configured to fit about a head 60 of the patient 25 (not shown in FIG. 2A), a traction motor 14 capable of exerting a traction load, an optional traction sling 16 that is sized and configured to fit about the cervical region 10 of the spine 8 of the patient 25, and a load transfer line 18 having a first end 19a capable of being placed in mechanical communication with the traction motor 14, and a second end 19b capable of being placed in mechanical communication with either the traction halter 12 or the traction sling 16. The load transfer line 18 is operative to transfer the traction load exerted by the traction motor 14 to the traction halter 12 or traction sling 16 to induce at least one of the extension posture and axially elongated posture in the cervical region 10 of the spine 8 of the patient 25.

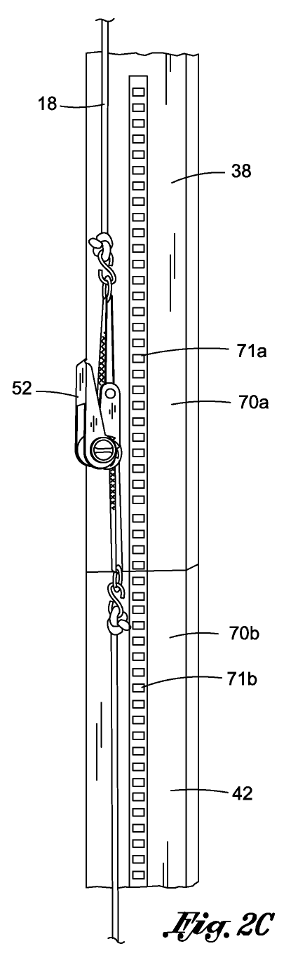

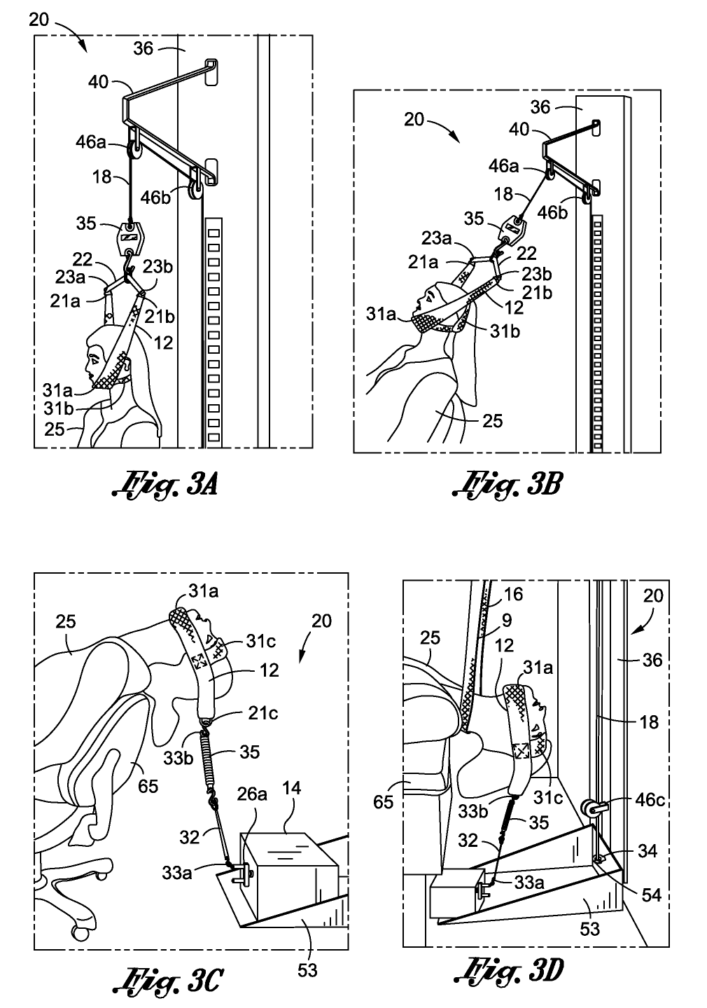

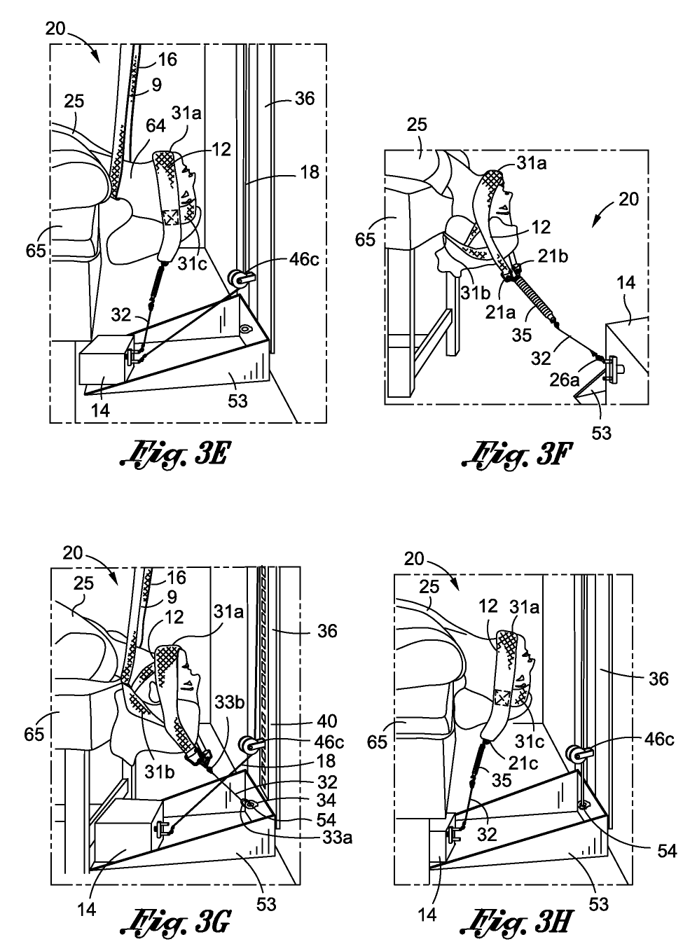

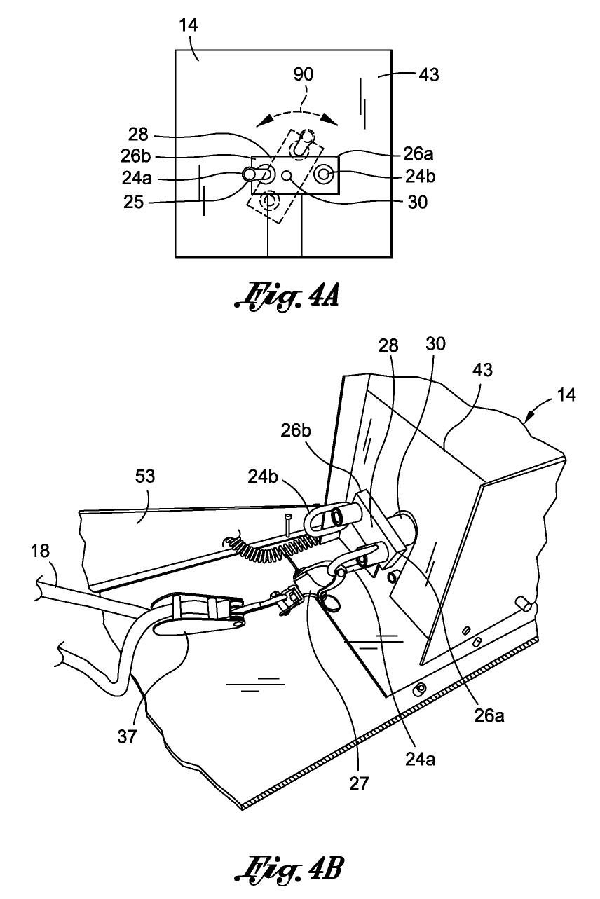

The traction apparatus 20 is thus capable of inducing a traction posture, such as a straight up-ward axial spinal traction, as shown in FIG. 3A, and also and/or alternatively an extension posture in the cervical region 10 of the spine 8, such as an axial extension or compression extension of the spine, by exerting a traction load that lifts and/or pulls one or more of the traction halter 12 and traction sling 16 and thereby stretches or pulls on the tissues and spinal region of the patient's neck 64. For example, as shown in FIG. 3A, an axially elongated posture can be induced in the cervical region 10 of the spine 8 by applying a traction load to pull the traction halter 12 straight upwardly in axially elongated spinal traction. As another example, in an axial extension of the cervical region 10 of the spine 8, a traction load can be exerted to pull the traction halter 12 in a direction that is axial and posterior to the longitudinal axis 16 of the spine, thereby distracting and extending the vertebrae of the spine, as shown for example in FIGS. 3B, 3F and 3G. In a compression extension of the cervical region 10 of the spine 8, a traction load can be exerted to pull the traction halter 12 in a rearward and even extreme rearward direction, thereby bending the neck backwards and inducing an anteriorly directed curve in the spine 8, as shown for example in FIGS. 3C, 3D and 3E. The traction sling 16 can be also be used in combination with the traction halter 12 to intensify the traction posture, for example by exerting an upwardly directed and/or transverse pull on the cervical region 10 of the spine 8, as shown for example in FIGS. 3D, 3E and 3G.

The traction halter 12 has a size and configuration that is selected to provide the desired traction treatment. For example, in the embodiment shown in FIG. 2B, the traction halter 12 can comprise a chin strap 31a adapted to be fitted under the chin 61 of the patient 25, and a back strap 31b that is adapted to be fitted about the back of the head 60 of the patient 25. The chin strap 31a and neck strap 31b support and hold the head 60 of the patient 25, and allow a traction load to be applied to the cervical region 10 of the patient's spine 8 by exerting a pull on the straps 31a, 31b that pulls the patient's head 60 in an angle and direction with respect to the longitudinal axis 16 of the patient's spine 8 that provides the desired treatment. In yet another version, as shown in FIG. 3C, the traction halter 12 comprises a chin strap 31a and a forehead strap 31c adapted to be fitted about the forehead 63 of the patient 25. In this version the chin strap 31a and forehead strap 31c similarly support and hold the patient's head 60 and allow a traction load to be applied by exerting a pull on the straps 31a, 31b. In particular, the halter 12 shown in FIG. 3C may be useful for embodiments in which the traction load is exerted to pull the patient's head 60 backwards and to induce a compression extension load into the cervical region 10 of the patient's spine 8.

Depending upon the type of traction being performed, the cervical traction apparatus 20 can also optionally comprise a traction sling 16 that fits about the cervical region 10 of the spine 8 of the patient 25 (i.e., about the patient's neck 64). The traction sling 16 can comprise at least one traction strap 9, which can be padded for the patient's comfort, and which is sized and configured to at least partially encircle a portion of the cervical region 10 of the spine 8, such as an upper, mid or lower region of the cervical spine. The traction strap 9 has sufficient strength to allow a portion of the cervical region 10 to be at least partially lifted when a traction load is applied to pull on the traction sling 16, preferably without excessive discomfort to the patient 25.

The traction apparatus 20 further comprises a load transfer line 18 that has a first end 19a that is capable of being placed in mechanical communication the traction motor 14, and a second end 19b that is capable of being placed in mechanical communication with at least one of the traction halter 12 and traction sling 16, and that is operative to transfer a traction load output from the traction motor 14 to the traction halter 12 and/or traction sling 16. For example, the load transfer line 18 may comprise one or more ropes, wires, and similar elements that are linked together to mechanically connect the motor 14 to the traction halter 12 and/or traction sling 16, the load transfer line 18 being capable of being disposed in tension with the traction halter 12 and/or traction sling 16 and the traction motor 14. A transfer of the traction load from the motor 14 can proceed by exertion of a traction load or traction force that pulls on the transfer line 18, the force of which pull is mechanically transmitted through the line 18 and to at least one of the traction halter 12 and traction sling 16. The transfer of this traction load pull thus results in a substantially simultaneous pull and/or lift on the region of the patient's body about which the traction halter 12 and/or traction sling 16 is fitted. For example, the transfer of the traction load results in a pull on the patient's head 60 when using the traction halter 12, and results in a pull on the patient's neck 64 in the case where the traction sling 16 is used. Conversely, a decrease in the traction load output by the motor 14 at least partially releases the load transfer line 18, and lessens the pull on the traction halter 12 and/or traction sling 16.

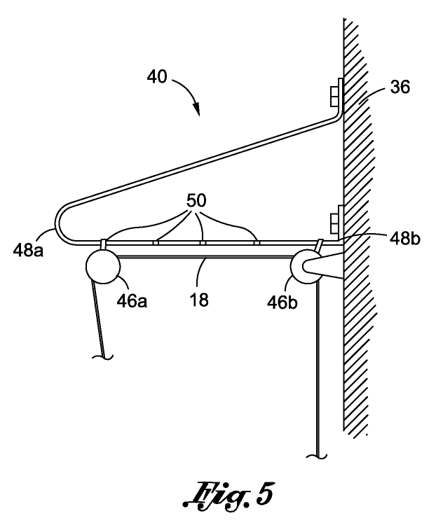

The load transfer line 18 may further comprise other elements in the line that aid in application of the traction load. For example, as shown in FIGS. 2A and 2B, the load transfer line 18 can comprise a scale 35, such as an in-line spring scale or a hanging digital tension scale, that allows measurement of the traction load being applied, and also modulates the traction load to provide a more continuous and comfortable onset of the traction load to the patient 25. Also, the hanging digital tension scale may act to protect the patient by having a tension “failure limit” beyond which the scale with initiate an audible alarm, thereby protecting the patient from excessive traction loads. The apparatus 20 can also optionally comprise a spreader bar 22, such as a horizontal or V-shaped spreader bar, that enhances the comfort and efficiency of the application of the traction load to the patient 25, by providing a more uniform application of the traction load to the patient, reducing squeezing or pinching of the patient by the traction halter straps 31a, 31b or traction sling strap 9, as well as by facilitating easy entry and exit of the patient into and out of the traction halter 12 and/or traction sling 16. In the version shown in FIG. 2B, the traction halter 12 comprises first and second halter ends 21a, 21b that are mechanically connected to first and second ends 23a, 23b of the spreader bar 22. In the version shown in FIG. 2A, the traction sling 16 comprises first and second sling ends 17a, 17b that are mechanically engaged to the first and second ends 23a, 23b of the spreader bar 22. In the versions shown in FIGS. 2A and 2B, the spreader bar 22 is inserted into the load transfer line 18 below the in-line scale 35, such as via a hook at the end of the scale 35 that is attached to the spreader bar 22 at about the middle of the length of the bar 22. The traction apparatus 20 can also comprise components such as at least one in-line strap ratchet winch mechanism 52 which may be a part of the load transfer line 18, and that can be used to increase or decrease tension in the line 18 just prior to or during cervical traction sessions. FIG. 2C shows an embodiment of a strap ratchet winch mechanism suitable for use with the cervical traction apparatus 20 of the invention. The traction apparatus 20 can also comprise a rope ratchet mechanism 37 that can be used to connect the load transfer line to the traction motor 14 and to increase and/or decrease tension in the line 18, as shown in FIG. 4B.

The cervical traction apparatus 20 further comprises a traction motor 14 that is capable of exerting the traction load having the magnitude that is intermittently varying, as described above. In one version, the traction motor 14 is capable of exerting the intermittent traction load by pulling on the line 18 connecting the motor 14 to the traction halter 12 and/or traction sling 16 with a force having a magnitude that is varied according to the desired intermittent traction load application. For example, the motor 14 may exert an intermittent force that alternately increases and decreases the pull on the line 18, thereby increasing or decreasing the tension in the line 18 and magnitude of the traction load being applied to the patient's body region. In one version, the traction motor 14 has an on-state in which the traction load is exerted at the intermittently varying magnitudes. The traction motor 14 may also have an off-state where either no traction load is exerted, or the traction load being exerted on the patient's body region is static and non-varying, depending upon the configuration of the apparatus 20.

An example of a traction motor 14 capable of providing such an intermittent traction force is a rotating motor or eccentric motor, as show for example in FIG. 4A. In this version, the rotating motor 14 comprises one or more hook-ups 24a, 24b, to which the line 18 can be mechanically engaged, such as via a swivel snap 27, which are disposed towards the distal ends 26a, 26b of a rotatable rod 28. The rotatable rod 28 is attached to a pin 30 that extends from within the motor casing 43, and that rotates upon activation of the motor 14. Activation of the rotating motor 14, for example by powering up via an external or internal power source (not shown), thus results in rotation of the pin 30 and simultaneous rotation of the rod 28 in a circular path 90 that is in a plane defined by the length of the rod 28, such as in a vertical plane in the version shown in FIG. 4A. The rotating movement of the rod 28 pulls the line 18 with a force that varies according to the position of the rod 28 in the circular path. For example, the force exerted on the line 18 is the least when the rod end 26a to which the line 18 is connected is rotated in a direction that decreases the tension in the line 18, and the force exerted is greatest when the rod end 26a is rotated in a direction that increases tension in the line 18. In the versions shown in FIGS. 3C and 3F, a lighter traction load is applied when the end 26a of the rod 28 is rotated in a direction that is towards a direction of travel of the line 18 away from the motor 14, such as in an upward direction towards the head 60 of the patient 25, and the load continuously increases throughout the rotation of the rod 28 until the heaviest load is achieved at the point where the rod end 26a is rotated furthest away from the direction of travel of the line 18 away from the motor 14, such as in a downwards direction away from the head 60 of the patient 25. Thus, by rotating the rod 28 in a circular motion, the traction force applied to the traction halter 12 and/or traction sling 16 and patient 25 can be intermittently decreased and increased in magnitude in a sinusoidal fashion. In yet another aspect, the rod 28 may be laterally off-set with respect to the position of the pin 30, and thus may comprise a more lateral hook-up 24b and a more medial hook-up 24a, either of which can be selected for connection to the line 18. The more lateral hook-up 24b carves out a circular path having a greater diameter, resulting in a greater difference between the maximum and minimum traction load magnitudes, whereas the more medial hook-up 24a carves out a smaller circular path, resulting in a smaller magnitude difference. Similarly, it can be understood that the length of the rod 28 can be selected according to the traction load differential that is desired. To inhibit the line 18 from twisting or knotting during the rotating operation of the motor 14, the motor can comprise hook-ups 24a, 24b that are swiveling hook-ups, capable of rotating independently of the rod 28. The line 18 can also be attached to the hook ups 24a, 24b via one or more snap swivels 27 that have a swiveling base that can rotate 180° independently of a clip portion that attaches to the hook-ups 24a, 24b, as shown for example in FIG. 4B. As is also shown in FIG. 4B, the line 18 can be connected to the snap swivel 27 via a rope ratchet 37, which can assist in maintaining the desired tension in the line 18 and can also be used to increase and/or decrease the tension in the line 18.

The cervical traction apparatus 20 can also optionally comprise a second load transfer line 32 having a first end 33a that is capable of being mechanically engaged, such as via the rope ratchet mechanism 37, to either the traction motor 14 or a second traction load source 34, and a second end 33b capable of being mechanically engaged to the traction halter 12 (or optionally to the traction sling 16), as shown for example in FIGS. 3C, 3D, 3E, 3F, 3G and 3H. Similarly to the first load transfer line 18, the second load transfer line is capable of being disposed in tension with (1) the traction motor 14 or second traction load source 34, and (2) the traction halter 12 or traction sling 16, to exert a traction load therebetween. The second load transfer line 32 may be used, for example, when the traction treatment involves the use of both the traction halter 12 and traction sling 16 to support and/or exert a traction load on the patient 25. The second traction load source 34 can comprise a source of static traction load, such as a static load adjustable via the rope ratchet mechanism 37, or may also be capable of exerting an intermittently varying traction load, similar to the traction motor 14. In one version, both the first and second load transfer lines 18, 32 are connected to the same traction motor 14, such as via the hook-ups 24a, 24b at the opposing ends 26a, 26b of the rotating rod 28. It should furthermore be understood that the first and second load transfer lines 18, 32 can be interchangeable with one another, such that for example the first load transfer line 18 may be connected to a static traction source while the second line 32 is connected to the traction motor 14, or vice versa, or alternatively both the first and second lines 18, 32 may be connected to the traction motor 14 or to separate motors. Thus, even though the first load transfer line 18 is described in the embodiments herein as the line supported by the traction frame 38, and the second load transfer line is referred to as the line being used in addition to said first line, it should be understood that the first and second lines 18, 32 are not limited to such embodiments, but rather that first and second lines 18, 32 can be employed in the traction apparatus 20 in any configuration suitable for achieving the desired cervical traction.

The traction apparatus 20 further comprises a traction support 36 that is configured to be capable of supporting and/or anchoring at least one of the first and second load transfer lines 18, 32 such that at least one of the traction halter 12 and traction sling 16 is pulled in a direction that induces the axially elongated and/or extension posture in the cervical region 10 of the spine 8 upon exertion of the traction load. In one version, the traction support 36 is configured to support and/or anchor at least one of the first and second load transfer lines 18, 32 such that the traction halter 12 and/or traction sling 16 are pulled upwardly upon exertion of a traction load. In the version shown in FIGS. 2A and 2B, the traction support 36 comprises an L-frame 38 having a base 53 and a beam 42 extending upwardly from an end of the base at a 90 degree angle. One or more of the base 53 and upwardly extending beam 42 can be secured to a wall and/or floor, for example in an examination room, or alternatively, the L-frame 38 may be free-standing. The L-frame 38 further comprises a laterally extending support arm 40, for example in the shape of a V-bar as shown in FIGS. 2A and 2B, that extends outwardly from an upper portion of the upwardly extending beam 42. The support arm 40 comprises one or more pulleys 46a, 46b mounted along a length of the support arm 40, through which the load transfer line 18 can be threaded, thereby supporting and anchoring the load transfer line 18 at a position above the patient 25. The load transfer line 18 threaded through the pulleys 46a, 46b is thus positioned to hang from above the patient 25, who can be seated or placed in a supine position below the support arm 40, such as on an examination chair or bench 65. In the version shown in FIG. 3A, the support arm 40 allows for the traction halter 12 to be pulled from substantially directly above the patient 25, which induces a substantially straight axial traction of the spine of the patient. In the version shown in FIG. 3D, the support arm 40 (not shown) allows the traction sling 16 to be pulled from substantially directly above the patient 25, thereby pulling the portion of the cervical region 10 about which the sling 16 is fitted in an upward direction. Also, as shown in FIG. 3B, the patient 25 and/or support arm 40 may be positioned and angled with respect to one another such that the traction force is exerted at an upwards and posterior angle producing an axial extension traction force.

In one version, the pulley 46a is slideably attachable to the support arm 40 so that it can be positioned at different lengths along the arm 40 and at different distances with respect to the other pulley 46b, thereby increasing or decreasing the angle of the traction load. For example, a very low angle of traction load can be achieved by positioning a first pulley 46a at the distal end 48a of the support arm 40, and positioning the second pulley 46b at the interior end 48b of the support arm 40, as shown for example in FIG. 5. The angle of pull can be progressively increased by sliding the first pulley 46a towards the second pulley 46b at the interior end 48b of the support arm 40. In one version, various stops are placed along the length of the support arm 40 to maintain the first and second pulleys 46a, 46b at desired positions. For example, the stops can comprise grooves 50 formed in the support arm 40 that are sized to hold one or more of the pulleys 46a, 46b within the groove. In one version, the support arm 40 comprises four such stops along its length, to provide four different angles of traction load.

The traction support 36 can further comprise one or more pulleys, hooks, D-rings, and similar parts that are capable of supporting the load transfer line 18 as it passes from the traction motor 14 or static traction load source to the traction halter 12 or traction sling 16. In the version shown in FIGS. 2A and 2B, the traction support 36 comprises a further pulley 46c affixed to a lower portion of the upwardly extending beam 42, through which the load transfer line 18 can be threaded. The position of the pulley 46c along the height of the beam 42 may also be adjustable, to allow selection of the desired high to low traction load intensity.

In a specific example of a suitable cervical traction apparatus 20, the traction support 36 comprises an upwardly extending beam 42 having two wall-mounted or free-standing steel face plates 70a, 70b with dimensions of 75″H×7″W×38″L. Two pieces of load-lock tracking 71a, 71b, having a working load capacity of 500 pounds, are mounted by nuts/bolts to the face plates 70a, 70b. For a free standing unit, the face plates 70a, 70b are attached by nuts/bolts to a 5′ upright support bar made of 2″×1″ square steel tubing (not shown). The lower face plate 70b is attached by nuts/bolts to a metal floor track 72 having dimensions of 6″H×11″W×29″, which makes up a part of the base 53. The floor track 72 can be made to allow the traction motor 14 to slide into it and be secured at numerous spots along the track 72 with screws. A support arm 40 in the shape of a traction V-bar, with two pulleys 46a, 46b having 60 lb capacity each is inserted into two tube-shaped metal receptacles 73a, 73b that are welded to the upper face plate 70a and screwed into place. A load-lock with a pulley 46c having a 282 lb capacity welded to it is inserted into the load-lock tracking 71a, 71b. A load transfer line 18 comprising a ¼″ polyester rope having a 150 lb capacity is strung through a ¼″ rope ratchet mechanism 37 having a 130 lb capacity, which is attached by a 1¾″ metal S-hook having a 110 lb load capacity to a metal snap swivel 27 having a 1650 lb capacity. The metal snap swivel 27 is attached to a rotating metal eye hook having a needle bearing insert that is screwed into a metal eccentric (rotating rod 28) that is mounted by a socket head set screw to the shaft of the traction motor 14. The traction motor 14 comprises a gear motor housed in a 6.5″H×8.625″W×11.25″L metal box casing 43 with a removable lid. The gear motor rotates at 12 revolutions per minute, and is further described in the manufacturer's literature entitled “Dayton Shaded Pole and Permanent Split Capacitor Type Gearmotors,” Form 8S706, by Dayton Electric Mfg. Co., 1996, and “Product Specific Information Manual for Dayton Shaded Pole Gearmotors” Form 5S3870 by Dayton Electric Mfg. Co., 1996, both of which are herein incorporated by reference in their entireties. The rope of the load transfer line 18 is strung through the load-lock pulley 46c, two traction V-bar pulleys 46a, 46b and then connected with a firm knot to an in-line, 66 lb scale 35 that can be attached by a metal S-hook having an 80 lb capacity to a traction spreader bar 22. A traction sling 16 made of foam padded 1″ polypropylene belting can be suspended by metal O-rings from the traction spreader bar 22. Alternatively, the traction halter 12 can be attached to the spreader bar 22 or scale 35. Also, the halter 12 can be attached to the lower face plate 70b and/or load-lock track 71b via the second load transfer line 32, comprising a ⅛″ polyester rope having an 80 lb. capacity, which is strung through a ⅛″ rope ratchet mechanism having a load capacity of 75 lbs (not shown) and attached at both ends by 1½″ metal S-hooks having a 55 lb. working capacity, or can alternatively be connected to the motor 14 by the same ⅛″ rope ratchet/metal S-hook system. While this specific embodiment is described for the purposes of further illustrating the invention, it should be understood that the cervical traction apparatus of the invention is not limited to the specific embodiments described herein, and also that the cervical traction method according to the invention could be performed with a different apparatus or apparatus having a different configuration than that specifically described.

Alternatively, the apparatus 20 can be used to provide traction without requiring support from the L-frame, for example as shown in FIGS. 3C and 3F. In this version, the load transfer line 32 is mechanically engaged to the traction motor 14 and passes substantially directly from the traction motor 14 to connect to the traction halter 12, i.e. without passing through pulleys, hooks, etc, mounted on the traction support 36. Such embodiments may be suitable, for example, when the traction motor 14 and patient 25 can be positioned with respect to one another to provide a desired angle and direction of traction pull on the patient 25.

In an example of a general mode of operation of the apparatus 20, the patient 25 is positioned in a supine or seated position adjacent the traction motor 14 and/or the traction support 36, such as on an examination/treatment table or examination/treatment chair 65. One or more of the traction halter 12 and traction sling 16 are fitted about the patient 25, such as by positioning the traction halter 12 about the head 50 of the patient 25, and/or by positioning the traction sling 16 about a portion of the cervical region 10 of the spine 8 of the patient 25 (i.e., about the patient's neck 64). The patient 25 may also be strapped down to oppose the applied traction force, or alternatively the weight of the patient's body may suffice to oppose the applied traction force. Also, supporting cushions or blocks can be provided to position portions of the patient's body according to the desired traction posture. The first load transfer line 18 is mechanically connected to the traction motor 14 as well as to the traction halter 12 or optionally the traction sling 16, and the tension in the load transfer line 18 is increased or decreased, such as via an in-line ratchet winch strap mechanism 52, and/or via a rope ratchet/metal S-hook mechanism, until a desired starting traction load is achieved. The traction motor 14 is typically maintained in the off-state while the patient is prepared for traction. The traction motor 14 can then be switched to the on-state to initiate the intermittent traction load application, such as by sinusoidally increasing and decreasing the pull on the transfer load line 18. The tension of the line 18 can also be further adjusted after activation of the motor 14. As a safety feature, the patient 25 can also be provided with a remote switch 93 that is in electrical communication with the motor 14, such as via an electrical cord 95, and that can be used to switch the motor 14 to the off-state according to the patient's needs.

The application of the traction load exerts a pull on the patient's head 60 via the traction halter 12, and/or the region of the cervical vertebrae about which the traction sling 16 is fitted, thereby inducing an extension and/or axial elongation in the cervical spine 8 of the patient. In particular, the traction load can be applied to pull the traction halter 12 and thereby the head 60 of the patient 25, in an upward direction, thereby inducing an axial traction/elongation in the cervical region of the spine that axially spreads the vertebrae apart from one another. The traction load can also be applied to pull the traction halter 12 and thereby the head 60 of the patient 25 at a posterior/rearward angle, such that the anterior tissues/vertebrae of the cervical region are stretched while the posterior tissues/vertebrae are compressed, resulting in an overall curved extension posture of the neck and cervical region of the spine. The direction and angle of the pull on the traction halter 12 can also be selected according to the desired curvature to be imparted to the cervical region, as is described in more detail below. Without being bound by any theory, it is believed that the inducement of these extension and/or elongation postures may result in musculoligamentous changes in the cervical region 10 of the spine 8 that can at least partially “remodel” the cervical region, and thereby provide improved posture and structural curvature, as well as pain relief.

The traction load can also optionally be applied to exert a transverse traction load on the cervical region 10 of the spine 8 via the traction sling 16. By “transverse traction load” it is meant that the traction load is applied in a direction having a vector component 11 that is perpendicular to the longitudinal axis 6 of the supine patient's spine, although the actual direction of the pull (the sum of the vector components) may be either perpendicular or at an angle to such perpendicularity. The application of this transverse traction load induces an extension posture in the cervical region 10 of the spine 8 of the patient, such as a posterior directed extension of the spine 8, which extension posture can at least partially restore the proper curvature of the cervical region 10 of the spine 8. In other words, the traction load is applied to the traction sling 16 to exert an outwardly and/or anterior directed pull, such as an upward pull, on the cervical region 10 of the supine patient's spine 8. A traction load applied to exert such a pull on the patient's body region can also be referred to as a transverse traction load. The combination of both the traction halter 12 and the traction sling 16 can provide the desired remodeling to the cervical region of the spine, both by exerting a directed pull from the upper end of the cervical region 10 (i.e. via the traction halter 12), as well as by selectively pulling on portions of the cervical region 10 such as the upper, mid and lower cervical regions.

Suitable embodiments of the invention are described in more detail below. In each of the embodiments, the intermittent traction load can be initially applied for a session of from about 3 to about 5 minutes, and the session can be subsequently repeated for increasing durations, such as up to 15-20 minutes per session, with a minimum recommended traction session time of ten minutes and a maximum recommended session time of twenty minutes. It is known from published ligamentous creep charts that ligamentous deformation drastically reduces after approximately 20 minutes. A recommended frequency of the treatments is at least two to three times per week. In initial sessions, a recommended maximum traction poundage may be from about 1 to about 12 lbs, such as from about 5 to about 15 lbs, which may be increased in subsequent sessions until either the patient's tolerance level or a maximum recommended amount of about 40 lbs is reached. A differential between the maximum traction poundage and minimum traction poundage may be from about 1 to about 20 pounds, such as from about 1 to about 10 lbs, and even from about 1 to about 7 pounds. The cervical traction method according to the instant invention can be performed to provide conservative treatment for painful cervicogenic conditions, including neck pain, disc problems, hypolordosis, kyphosis, and other posture problems related to irregular curvature of the spine, by stretching the spinal tissue, reducing the pressure on intervertebral discs and the vertebral osseous bodies, as well as by effecting restoration of cervical lordosis and reduction of excessive anterior/posterior head carriage translation.

FIG. 3A shows an embodiment of a method and configuration of the apparatus 20 that provides seated axial elongation/distraction of the cervical region 10 of the patient's spine 8. In this version, the patient 25 is placed in a seated position on a treatment or examination chair 65, and a traction halter 12 having a chin strap 31a and back strap 31b are fitted about the head 60 of the patient 25. The first and second ends 21a, 21b of the traction halter 12 are mechanically engaged to first and second ends 23a, 23b of a horizontal spreader bar 22, which is connected via the spring-scale or digital scale 35 to the rest of the load transfer line 18. The load transfer line 18 is anchored to the traction support 36 above the patient's head via pulleys 46a, 46b, such that the load transfer line extends from the support arm 40 of the traction support 36 to the traction halter 12 in a direction that is axial with respect to the longitudinal axis 6 of the patient's spine 8, and is passed along the traction support 36 to the traction motor 14, where the transfer line 18 may be engaged to the traction motor 24 via the more lateral hook-up 24b. The traction load output by the traction motor 14 thus exerts a pull on the chin 61 and the back of the head 60 of the patient 25 that lifts and pulls on the patient's head 60, thereby elongating, distending and/or separating the vertebrae of the cervical region 10 of the spine 8 and inducing an axially elongated posture therein. This version of axial distraction treatment may be desirable for treatment in instances where all of the cervical spinal segments are positioned forward of a desired spinal arch line, and/or the upper and lower cervical spine has increased curve, as well as for the treatment of degenerated invertebral discs with associated decreased disc height and/or possible bulging of one or more of the disc(s).

FIG. 3B shows yet another embodiment of a method and configuration of the apparatus 20 to provide seated axial extension/distraction of the cervical region 10 of the patient's spine 8 by pulling in a rearward direction. In this version, similar to the version described for FIG. 3A above, the patient is placed in a seated position in a treatment or examination chair 65, and a traction halter 12 having a chin strap 31a and back strap 31b are fitted about the head 60 of the patient 25. The first and second ends 21a, 21b of the traction halter 12 are mechanically engaged to first and second ends 23a, 23b of a horizontal spreader bar 22, which is connected via the spring-scale or digital scale 35 to the rest of the load transfer line 18. The load transfer line 18 is anchored to the traction support 36 above the patient's head via pulleys 46a, 46b. However, in this embodiment the patient is positioned such that the load transfer line 18 extends in a direction that is at a rearward angle with respect to the longitudinal axis 6 of the spine 8 of the patient 25, from the traction halter 12 to the support arm 40 of the traction support 36. For example, the patient 25 can be positioned slightly forward from the support arm 40 and pulley 46a of the traction support 36, or alternatively the pulleys 46a, 46b could be positioned to anchor the transfer line 18 at a point on the support arm 40 behind the patient 25, to give the rearward angle. The rearward angle is desirably selected such that an axial extension posture is induced in the cervical region 10 of the patient's spine 8 upon exertion of the traction load. Similarly to the embodiment described above, the load transfer line 18 is passed along the traction support 36 to the traction motor 14, where the transfer line 18 may be engaged thereto via the more lateral hook-up 24b. The traction load output by the traction motor 14 thus exerts a pull on the chin 61 and the back of the head 60 of the patient, which lifts and pulls the patient's head in the rearward direction, thereby extending the lower vertebrae of the cervical region 10 of the spine 8 and inducing an extension posture. This version of axial extension treatment may be desirable in instances where all or most of the segments of the cervical region 10 of the spine 8 are forward of a desired spinal arch line, and no segments are behind the desired arch line, such as where the upper cervical spine has increased curve and the lower cervical spine has decreased curve.

FIG. 3F shows yet another embodiment of a method and configuration of the apparatus 20 to provide axial extension/distraction of the cervical region 10 of the patient's spine 8 by pulling in a rearward direction. In this version, the patient is placed in a supine position, such as on a treatment or examination bench 65, and the traction halter 12 having the chin strap 31a and back strap 31b are fitted about the head 60 of the patient 25. In this version, the first and second ends 21a, 21b of the traction halter 12 are mechanically engaged directly to the in-line spring scale or digital scale 35 and then to the rest of the load transfer line 32. The load transfer line 32 is passed substantially directly to the traction motor 14, i.e. without anchoring or passing the load transfer line onto or through a traction support 36. In this embodiment, the patient and the traction motor 14 are positioned with respect to one another such that the load transfer line 32 extends in a direction that is at a rearward angle with respect to the longitudinal axis 6 of the spine 8 of the patient 25 from the traction halter 12 to the traction motor 14, similarly to the seated rearward axial extension described above. For example, the patient can be positioned such that the head is elevated above the motor 14, and the motor 14 can be placed behind the patient 25, such that a transfer line 32 passes downwardly from the patient 25 to the motor 14 at the rearward angle. The rearward angle is desirably selected such that an axial extension posture is induced in the cervical region 10 of the patient's spine 8 upon exertion of the traction load. In this version, the transfer line 32 may be engaged to the traction motor 14 via the more medial hook-up 24a. The traction load output by the traction motor 14 thus exerts a pull on the chin 61 and the back of the head 62 of the patient 26, which pulls the patient's head 60 in the rearward direction, thereby extending the lower vertebrae of the cervical region 10 of the spine 8 and inducing an extension posture. This version of supine axial extension treatment may be desirable in instances where all or most of the vertebrae segments in the cervical region 10 are forward of the desired arch line, and no segments are behind the desired arch line, and where the upper and lower cervical spine has decreased curve. The treatment in this instance may also be aided by positioning a firm cervical roll-shaped pillow beneath the lower cervical segments of the spine 8.

FIGS. 3C and 3H show embodiments of a method and configuration of the apparatus 20 that provides seated or supine compression extension of the cervical region 10 of the patient's spine 8. In this version, the patient 25 is placed in a seated position (FIG. 3C) or supine position (FIG. 3H) on a treatment or examination chair or bench 65, and the traction halter 12 having a chin strap 31a and forehead strap 31c are fitted about the head 60 of the patient 25. In this version, the traction halter 12 has a top connecting end 21c that is connected directly to the in-line spring scale or digital scale 35 and then to the rest of the load transfer line 32. The load transfer line 32 can be passed substantially directly to the traction motor 14, i.e. without anchoring or passing the load transfer line 32 onto or through the traction support 36. In this embodiment, the patient and the traction motor 14 are positioned with respect to one another such that the load transfer line 32 extends in a direction that is at a rearward angle with respect to the longitudinal axis of the spine of the patient, similarly to the supine rearward axial extension method described above. However, in this instance, the rearward angle is more extreme than that selected in the axial extension methods, and is instead selected to induce a compression extension in the cervical region 10 of the spine 8 of the patient 25. For example, the patient 25 can be positioned such that the head is elevated above the motor 14, and the motor 14 can be placed behind the patient (FIG. 3C) or even under the patient (FIG. 3H), such that a transfer line passing downwardly from the patient 25 to the motor 14 has the rearward compression extension angle. In this version, the transfer line 32 may be engaged to the traction motor 14 via the more medial hook-up 24a. The traction load output by the traction motor 14 thus exerts a pull on the chin 61 and the forehead 63 of the patient 25, which pulls the patient's head 60 in a downward and rearward direction, thereby compressing the vertebrae together at the back of the spine 8 and extending the vertebrae apart at the front of the spine 8 in the cervical region 10. The seated version of this compression extension treatment may be desirable in instances where the upper cervical segments are forward of the desired arch line, the lower cervical segments are on or are forward of the desired arch line, and the upper cervical spine has decreased curve. The supine version of this compression treatment may be desirable in instances where all or most of the cervical segments are forward of the desired arch line and no segments are behind the desired arch line, and where the upper and lower cervical spine has decreased curve.

FIG. 3D shows yet another version of a method of traction treatment in which the use of the traction halter 12 and the traction sling 16 is combined to provide synergistic, two-directional compression extension traction of the cervical region 10 of the spine 8. In this embodiment, the patient is placed in a supine position and the load transfer line 32, traction halter 12 and traction motor 14 are configured with respect to one another as described and shown for the supine compression extension above in FIG. 3H. However, in addition to the above-described supine compression extension method and configuration is added the use of the traction sling 16, which is fitted about the neck 64 of the patient 25, such as about the upper, lower or mid cervical regions 10 of the neck 64, the first and second ends 17a, 17b of the traction sling 16 being mechanically engaged to the first and second ends 23a, 23b of the spreader bar 22. The spreader bar 22 is connected to a second in-line spring scale or digital scale 35, which is connected to a second load transfer line 18. In this version, the second load transfer line 18 is anchored to the traction support 36 above the patient's head via pulleys 46a, 46b, such that the second load transfer line 18 extends from the support arm 40 of the traction support 36 to the traction sling 16 in a direction that is transverse with respect to the longitudinal axis of the supine patient' spine (i.e., in a direction having an upwardly directed component). The second load transfer line 18 and is passed along the traction support 36, optionally via an in-line ratchet winch strap mechanism 52 (not shown in FIG. 3D), and to an eye hook 54 on the floor base 53 of the traction support 36, where the second load transfer line 18 is attached, such as by a metal S-hook (not shown), at a desired tension. In this version, the traction support 36 serves as a second traction load source 34 capable of providing a static traction load, as the support resists the pull induced by the tension in the second load transfer line 18. The traction load exerted by the traction sling 16 lifts the region of the cervical spine about which it is fitted, thereby inducing an extension posture in the vertebrae of the cervical region 10 of the spine 8. The extension posture induced by the traction sling 16 coupled with the compression extension induced by the traction halter 12 works synergistically to treat various problems related to irregular curvature of the spine. For example, the method can be used for treatment in instances where the upper cervical segments are forward of the desired arch line, the upper cervical spine has a decreased curve, and the lower cervical spine has either a decreased curve or a normal curve and is on the desired arch line. In the case where the lower cervical spine has decreased curve, the traction sling 16 is positioned and the second load transfer line 18 is angled such that it provides a transverse/anterior pull into the area of maximum curvature loss. In the case where the lower cervical spine has a normal curve, the cervical sling 16 is used at an intensity that is sufficient to hold the lower cervical segments in their normal position. If a second in-line scale 35 is used on the second load transfer line 18, it is preferably a digital scale rather than a spring scale in order to provide the necessary fulcrum stability of the traction sling 16.

FIG. 3E shows yet another version of a method of traction treatment in which the use of the traction halter 12 and the traction sling 16 is combined to provide two-directional synergistic compression extension traction of the cervical region of the spine. In this embodiment, the patient is placed in a supine position and the load transfer line 32, traction halter 12 and traction motor 14 are configured with respect to one another as described and shown for the supine compression extension as shown above in FIG. 3D. The traction sling 16 is fitted about the neck 64 of the patient 25, and is engaged to a second load transfer line 18 that is anchored from above the patient to the traction support 36, via pulleys 46a, 46b, as in the synergistic supine, two-way compression extension method described above. However, in this method, the second load transfer line 18 is passed along the traction support 36, through the pulley 46c located on the support beam 42, and is connected to the same traction motor 14 to which the first load transfer line 32 is connected. In this way, the traction motor 14 is capable of outputting a traction load having an intermittently varying magnitude to both the traction halter 12 and traction sling 16, such that a rearward pull on the head of the patient via the traction halter 12 is simultaneously accompanied by an upward pull on the neck 64 of the patient 25 via the traction sling 16, and a reduction in the magnitude of the traction force output by the traction motor 14 results in a simultaneous easing of the pull on the head 60 and neck 64 of the patient 25.

The transverse extension traction force induced by the traction sling 16 coupled with the compression extension traction force induced by the traction halter 12 as shown in FIG. 3E works synergistically to treat various problems related to irregular curvature of the spine 8. For example, the method can be used for treatment in instances where the upper cervical segments are forward of the desired arch line, the lower cervical segments are on or behind the desired arch line, the upper cervical spine has decreased curve, and the lower cervical spine has decreased curve. In this instance, the traction sling 16 should be positioned to provide a transverse/anterior pull that into the cervical area of maximum curvature loss. In yet another instance, treatment can be provided when all or most of the C6 to C7 segments are behind the desired arch line, and the upper and lower regions of the cervical spine have decreased curve. In this instance, the traction sling 16 is again positioned to provide a transverse anterior pull into the cervical region of maximum curvature loss.

A final version of a method of treatment is shown in FIG. 3G. This version provides synergistic use of the traction sling 16 and the traction halter 12 to provide cervical two-way axial extension traction while the patient 25 is in a supine position. In this version, the first load transfer line 18 is mechanically engaged to the traction sling 16 fitted about the back of the patient's neck 64 and to the traction motor 14, similar to the configuration of the load transfer line 18 in the supine two-way compression extension method as described above and shown in FIG. 3E. However, in this method, the traction halter 12 having the chin strap 31a and back strap 31b is connected via the second load transfer line 32 and a rope ratchet mechanism (not shown) to a second load source 34 that exerts a static load to provide a substantially static tension in the second load transfer line. For example, the second load transfer line 32 can be fixed to vertical, variably located load-lock ring (not shown in FIG. 3G) or other element on the traction support 36, such as on a bottom portion of the upwardly extending load-lock track 71b or to the eye-hook 54 on the floor base 53 of the traction support 36. Preferably, the second load transfer line 32 has a first end 33a that is connected to the traction support 36 such that the second load transfer line 32 passes from the traction halter to the traction support 36 at an angle and direction selected to provide the desired axial extension, such as the axial extension angles as described above for FIGS. 3B and 3F. In this way, the intermittent traction load is applied in a transverse direction to the patient's cervical spine region, while a static traction load is applied to exert the axial extension/distraction of the cervical region 10. The first load transfer line 18 can be connected to the more lateral motor hook-up 24b in this embodiment, to effect a greater change in the high to low magnitude of the applied traction load. This method of treatment can be used in the instance where the lower cervical segments are behind the desired arch line, the upper cervical segments are forward of the desired arch line, the upper cervical spine has increased curve, and the lower cervical spine has decreased curve. In this instance, a low to extra low setting should be selected for the traction sling 16.

Additional modifications and improvements of the present invention may also be apparent to those of ordinary skill in the art. Thus, the particular combination of components and steps described and illustrated herein is intended to represent only certain embodiments of the present invention, and is not intended to serve as limitations of alternative devices and methods within the spirit and scope of the invention. Along these lines, it should be understood that the traction support 36, traction sling 16, traction motor 14 and traction load transfer lines 18, 32 may take any of a variety of forms that are known or later developed in the art, and further contemplates that such existing or newly formed traction components, such as newly formed traction frames 38 and traction halters 12 and slings 16, should fall within the scope of the present invention. Also, it should be understood that the traction apparatus 20 can comprise other configurations, and can be used to perform traction methods other than those specifically described, and similarly the cervical traction method described herein can be performed with traction apparatus 20 other than those specifically described.

What is claimed is:

1. A method of treating irregularities in the lateral curvature of a cervical region of a spine in a patient in need thereof, the method comprising the steps of:
(a) placing the patient in a supine or seated position;
(b) positioning a traction halter about the head of the patient;
(c) optionally, positioning a traction sling about the cervical region of the patient; and
(d) exerting a traction load on one or more of the sling or halter to induce at least one of an extension posture and axially elongated posture in at least a portion of the cervical region of the spine,
wherein inducing at least one of the extension posture and axially elongated posture at least partially restores or diminishes the lateral curvature of the cervical region of the spine in the patient.

2. A method according to claim 1, wherein the traction load comprises an intermittently varying magnitude.

3. A method according to claim 2, wherein the intermittently varying magnitude comprises a sinusoidally varying magnitude.

4. A method according to claim 2, wherein the intermittently varying magnitude comprises a maximum magnitude and a minimum magnitude, and wherein a difference between the maximum and minimum magnitudes of the transverse traction load is from about 1 lb to about 20 lbs.

5. A method according to claim 4, wherein the difference is from about 1 lb to about 7 lbs.

6. A method according to claim 2, wherein the intermittently varying magnitude varies with a frequency of from about 5 cycles/min to about 20 cycles/min.

7. A method according to claim 2, wherein the intermittent traction load is a non-zero load.

8. A method according to claim 1 wherein (a) comprises placing the patient in a seated position, and (d) comprises exerting the traction load to pull the traction halter in a direction that is axial with respect to the longitudinal axis of the spine of the patient, and thereby inducing an axially elongated posture in the cervical region of the spine of the patient.

9. A method according to claim 1 wherein (a) comprises placing the patient in a seated or supine position, and (d) comprises exerting the traction load to pull the traction halter in a direction that is at a rearward angle with respect to the longitudinal axis of the spine of the patient, the rearward angle being selected to induce an axial extension posture in the cervical region of the spine of the patient.

10. A method according to claim 1, wherein (a) comprises placing the patient in a seated or supine position, and (d) comprises exerting the traction load to pull the traction halter in a direction that is at an extreme rearward angle with respect to the longitudinal axis of the spine of the patient, the extreme rearward angle being selected to induce a compression extension posture in the cervical region of the spine of the patient.

11. A method according to claim 10, wherein (a) comprises placing the patient in a supine position, step (c) is performed to position a traction sling about the cervical region of the patient, and wherein (d) further comprises exerting a second traction load on the traction sling to pull the cervical region of the spine in a transverse direction with respect to a longitudinal axis of the spine.

12. A method according to claim 11, wherein the second traction load exerted on the traction sling comprises an intermittently varying magnitude.

13. A method according to claim 1, wherein step (a) comprises placing the patient in supine position, step (c) is performed to position a traction sling about the cervical region of the patient, and wherein step (d) comprises exerting the traction load on the traction sling to pull the cervical region of the spine in a transverse direction with respect to a longitudinal axis of the spine, the traction load comprising an intermittently varying magnitude, and further exerting a second traction load on the traction halter at a rearward angle with respect to the longitudinal axis of the spine of the patient, the rearward angle being selected to induce an axial extension posture in the cervical region of the spine of the patient, the second traction load being a substantially static traction load.

14. A method according to claim 1 wherein the patient is suffering from at least one of headaches, neck pain, upper back pain, disc problems, spondylosis, hypolordosis and kyphosis with or without associated postural abnormalities.

15. A traction apparatus for effecting traction of a cervical region of a spine of a patient, the apparatus comprising:
(a) a traction halter that is sized and configured to fit about a head of a patient;
(b) a traction motor capable of exerting a traction load;
(c) optionally, a traction sling that is sized and configured to fit about the cervical region of the spine of the patient; and
(d) a load transfer line having a first end capable of being place in mechanical communication with the traction motor and a second end capable of being placed in mechanical communication with either the traction halter or the traction sling, the load transfer line being capable of transferring the traction load from the traction motor to the traction halter or traction sling to induce at least one of an extension posture and axially elongated posture in the cervical region of the spine.

16. A traction apparatus according to claim 15, wherein the traction motor is capable of exerting a traction load having a magnitude that is intermittently varying.

17. A traction apparatus according to claim 15, wherein the traction motor is capable of exerting a traction load having a sinusoidally varying magnitude.

18. A traction apparatus according to claim 15, wherein the traction motor is capable of exerting an intermittently varying magnitude comprising a maximum magnitude and a minimum magnitude, and wherein a difference between the maximum and minimum magnitudes of the transverse traction load is from about 1 lb to about 20 lbs.

19. A traction apparatus according to claim 18, wherein the difference is from about 1 lb to about 7 lbs.

20. A traction apparatus according to claim 15, wherein the traction motor is capable of exerting an intermittently varying magnitude that varies with a frequency of from about 5 cycles/min to about 20 cycles/min.

21. A traction apparatus according to claim 15, wherein the traction motor is configured to have an on-state and an off-state, and wherein in the on-state the traction motor is capable of exerting an intermittent traction load that is a non-zero load.

22. A traction apparatus according to claim 15, wherein the second end of the load transfer line is in mechanical communication with the traction halter, the traction halter comprising a chin strap configured to be fitted underneath the chin, and a forehead strap configured to be fitted about the forehead, and wherein the load transfer line extends from the traction motor to the traction halter at an angle selected such that exerting the traction load pulls the traction halter at an extreme rearward angle with respect to the longitudinal axis of the spine of the patient, the extreme rearward angle being selected to bend the head of the patient backwards and thereby induce a compression extension posture in the cervical region of the spine of the patient.

23. A traction apparatus according to claim 15, wherein the second end of the load transfer line is in mechanical communication with the traction halter, the traction halter comprising a chin strap configured to be fitted underneath the chin, and a back strap configured to be fitted about the back of the head, and wherein the load transfer line extends from the traction motor to the traction halter at an upward angle selected such that exerting the traction load pulls the traction halter in a direction that is axially parallel to the longitudinal axis of the spine of the patient, the upward angle being selected to induce an axially elongated posture in the cervical region of the spine of the patient.

24. A traction apparatus according to claim 15, wherein the second end of the load transfer line is in mechanical communication with the traction halter, the traction halter comprising a chin strap configured to be fitted underneath the chin, and a back strap configured to be fitted about the back of the head, and wherein the load transfer line extends from the traction motor to the traction halter in a direction that is at a rearward angle with respect to the longitudinal axis of the spine of the patient, the rearward angle being selected to induce an axial extension posture in the cervical region of the spine of the patient.

25. A traction apparatus according to claim 15 further comprising:
(e) a traction support comprising an L-frame having a laterally extending support arm, the support arm being configured to be capable of anchoring the load transfer line at a position above the patient, such that at least one of the traction halter and traction sling is pulled upwardly upon exertion of the traction load.

26. A traction apparatus according to claim 25, wherein the second end of the load transfer line is in mechanical communication with the traction halter, the traction halter comprising a chin strap configured to be fitted underneath the chin, and a back strap configured to be fitted about the back of the head, and wherein the traction support anchors the load transfer line at a position from substantially above the patient such that the load transfer line extends from the traction support to the traction halter in a direction that is axial with respect to the longitudinal axis of the spine of the patient, the axial direction being selected to be capable of inducing an axially elongated posture in the cervical region of the spine of the patient upon exertion of the traction load.

27. A traction apparatus according to claim 25, further comprising:
(f) a second load transfer line having a first end capable of being placed in mechanical communication with at least one of the traction motor and a second traction load source capable of exerting a second traction load, and a second end capable of being placed in mechanical communication with either the traction sling or the traction halter, the second load transfer line being configured to transfer a traction load from either the second traction load source or the traction motor to the traction sling, wherein the traction support is further configured to be capable of anchoring the second load transfer line at the position above the patient.

28. A traction apparatus according to claim 27 wherein (1) the second end of the load transfer line is in mechanical communication with the traction halter, the traction halter comprising a chin strap configured to be fitted underneath the chin of the patient, and a forehead strap configured to be fitted about the forehead of the patient, (2) the load transfer line extends from the traction motor to the traction halter at an angle selected such that exerting the traction load pulls the traction halter at an extreme rearward angle with respect to the longitudinal axis of the spine of the patient, the extreme rearward angle being selected to induce a compression extension posture in the cervical region of the spine of the patient, the traction motor exerting an intermittent traction load, and wherein (3) the second end of the second load transfer line is in mechanical communication with the traction sling, and the first end of the second load transfer line is in mechanical communication with the second traction load source, the second traction load source exerting a static traction load, and wherein the traction support anchors the second load transfer line at a position that is substantially directly above the supine patient to pull the cervical region of the spine in a transverse direction with respect to a longitudinal axis of the spine upon exertion of the static traction load.

29. A traction apparatus according to claim 27 wherein (1) the second end of the load transfer line is in mechanical communication with the traction halter, the traction halter comprising a chin strap configured to be fitted underneath the chin of the patient, and a forehead strap configured to be fitted about the forehead of the patient, (2) the load transfer line extends from the traction motor to the traction halter at an angle selected such that exerting the traction load pulls the traction halter at an extreme rearward angle with respect to the longitudinal axis of the spine of the patient, the extreme rearward angle being selected to induce a compression extension posture in the cervical region of the spine of the patient upon exertion of the traction load from the traction motor, the traction motor exerting an intermittent traction load, and (3) the second end of the second load transfer line is in mechanical communication with the traction sling, and the first end of the second load transfer line is in mechanical communication with the traction motor, and wherein the traction support anchors the second load transfer line from a position that is substantially directly above the supine patient to pull the cervical region of the spine in a transverse direction with respect to a longitudinal axis of the spine upon exertion of the intermittent traction load.

30. A traction apparatus according to claim 27 wherein (1) the second end of the second load transfer line is in mechanical communication with the traction halter, wherein the traction halter comprises a chin strap configured to be fitted underneath the chin of the patient, and a back strap configured to be fitted about the back of the head, and the first end of the second load transfer line is in mechanical communication with the second traction load source, the second traction load source being capable of exerting a static traction load, (2) the second load transfer line extends from the static load source to the traction halter at a rearward angle with respect to the longitudinal axis of the spine of the patient, the rearward angle being selected to be capable of inducing an axial extension posture in the cervical region of the spine of the patient upon exertion of the static traction load, and (3) the first end of the load transfer line is in mechanical communication with the traction motor, the traction motor being capable of exerting an intermittent traction load, and the second end is in mechanical communication with the traction sling, and wherein the traction support anchors the load transfer line from a position substantially directly above the supine patient to pull the cervical region of the spine in a transverse direction with respect to a longitudinal axis of the spine upon exertion of the intermittent traction load.
